(19) United States
(12) Patent Application Publication
(10) Pub. No.: US 2023/0204601 A1
(43) Pub. Date: Jun. 29, 2023

(54) SYSTEMS AND METHODS FOR DETECTION OF LAMININ BETA-1 SUBUNIT WITHIN TISSUE

(71) Applicant: Axogen Corporation, Alachua, FL (US)

(72) Inventors: Kasra TAJDARAN, Alachua, FL (US); Mariska Anouska Ter HAAK, Summit, NJ (US)

(73) Assignee: Axogen Corporation, Alachua, FL (US)

(21) Appl. No.: 18/145,873

(22) Filed: Dec. 23, 2022

Related U.S. Application Data
(60) Provisional application No. 63/294,083, filed on Dec. 28, 2021.

(51) Int. Cl. G01N 33/68; G01N 33/543

(52) U.S. Cl. CPC G01N 33/6887; G01N 33/54386; G01N 2333/78; G01N 2470/04

(57) ABSTRACT

A method for performing an enzyme-linked immunosorbent assay (ELISA), the method comprising immobilizing a capture antibody to a substrate, wherein the capture antibody is configured to bind to a laminin beta-1 chain; adding a micronized tissue sample to the substrate containing the capture antibody; adding a detection antibody to the substrate containing the capture antibody and the micronized tissue sample, wherein the detection antibody is configured to bind to a laminin gamma-1 chain; detecting whether a complex of the capture antibody, a target antigen in the micronized tissue sample, and the detection antibody has been formed, wherein presence of the complex indicates presence of the target antigen with intact tertiary structure in the micronized tissue sample, and absence of the complex indicates absence of the target antigen with intact tertiary structure in the micronized tissue sample.

### CROSS-REFERENCE TO RELATED APPLICATIONS

This patent claims the benefit of priority under 35 U.S.C. § 119 to U.S. Provisional Pat. Application No. 63/294,083, filed Dec. 28, 2021, which is herein incorporated by reference in its entirety.

### TECHNICAL FIELD

The present disclosure generally relates to the field of biochemistry assays, and more particularly, relates to evaluating the biological activity of tissue.

### BACKGROUND

Enzyme-Linked Immunosorbent Assay (ELISA) is a commonly used analytical biochemistry assay to detect the presence of a target antigen in a sample. The target antigen may be any soluble substance, such as peptides, proteins, antibodies, and hormones. In human tissue, laminin proteins are considered as the basement membrane proteins that play an important role in cellular function and tissue morphogenesis. Unfortunately, commercially available ELISA kits are unable to specifically identify active laminin isoforms with beta-1 subunits. As a result, with currently available tools, it may not be possible to assess the presence and concertation of the active specific laminin isoforms with the beta-1 subunit within a given tissue sample. Improved systems and methods for the analysis of human active laminin isoforms in tissues, such as nerve tissues, are thus needed.

### SUMMARY

In at least one aspect, the present disclosure provides a method of performing an enzyme-linked immunosorbent assay (ELISA), the method comprising immobilizing a capture antibody to a substrate, wherein the capture antibody is configured to bind to a laminin beta-1 chain; adding a micronized tissue sample to the substrate containing the capture antibody; adding a detection antibody to the substrate containing the capture antibody and the micronized tissue sample, wherein the detection antibody is configured to bind to a laminin gamma-1 chain; detecting whether a complex of the capture antibody, a target antigen in the micronized tissue sample, and the detection antibody has been formed, wherein presence of the complex indicates presence of the target antigen with intact tertiary structure in the micronized tissue sample, and absence of the complex indicates absence of the target antigen with intact tertiary structure in the micronized tissue sample.

Various embodiments of the method may include one or more of the following aspects. The target antigen may include one or more of laminin 111 (α1β1γ1), laminin 211 (α2β1γ1), laminin 311 (α3β1γ1), laminin 411 (α4β1γ1), and laminin 511 (α5β1γ1). The detection step may comprise determining a concentration of the target antigen. Passing or failing the micronized tissue sample based at least in part on the determined concentration of the target antigen. The detection step may comprise determining a presence of the target antigen. Passing or failing the micronized tissue sample based at least in part on the determined presence of the target antigen. Washing the substrate with a buffer solution chosen from tris-buffered saline, tris-buffered saline and polysorbate 20, phosphate-buffered saline, or mixtures thereof, after one or more of: adding the capture antibody to the substrate, adding the micronized tissue sample to the substrate, or adding the detection antibody to the substrate. The detection step may be performed using one or more of visual inspection, a spectrophotometer, or a microplate reader. Incubating the substrate after one or more of: adding the capture antibody to the substrate, adding the micronized tissue sample to the substrate, or adding the detection antibody to the substrate. The tissue sample may be nerve tissue. The tissue sample may include one or more of human epithelial tissue, connective tissue, muscular tissue, vascular tissue, dermal tissue, skeletal tissue, cardiac tissue, urological tissue, skin tissue, dura tissue, intestinal tissue, gingiva tissue, or adipose tissue.

The present disclosure also provides a method of detecting an active laminin protein in a tissue sample, the method comprising preparing a micronized mixture from a tissue sample, wherein the tissue sample contains nerve tissue; immobilizing a first antibody to the substrate; adding the micronized mixture to the substrate containing the first antibody, wherein the first antibody is configured to bind to a first antigenic site of the laminin protein if present in the micronized mixture; adding a second antibody to the substrate containing the first antibody and the micronized mixture, wherein the second antibody is configured to bind to a second antigenic site of the laminin protein if present in the micronized mixture; and detecting whether a complex of the first antibody, the laminin protein, and the second antibody has been formed, wherein presence of the complex indicates presence of one or more of laminin 111 (α1β1γ1), laminin 211 (α2β1γ1), laminin 311 (α3β1γ1), laminin 411 (α4β1γ1), and laminin 511 (α5β1γ1) isoforms with intact tertiary structure, and absence of the complex indicates absence of one or more of laminin 111 (α1β1γ1), laminin 211 (α2β1γ1), laminin 311 (α3β1γ1), laminin 411 (α4β1γ1), and laminin 511 (α5β1γ1) with intact tertiary structure.

Various embodiments of the method may include one or more of the following aspects. The first antigenic site may be a beta-1 laminin subunit of the laminin protein. The second antigenic site may be a gamma-1 laminin subunit of the laminin protein. The first antibody may be an anti-laminin beta-1 antibody. The anti-laminin beta-1 antibody may be a mouse anti-human laminin beta-1 monoclonal antibody. The second antibody may be an anti-laminin gamma-1 antibody. The anti-laminin gamma-1 antibody biotinylated anti-h/r/ laminin gamma-1 Purified Mouse Monoclonal IgG. Preparing and analyzing laminin reference standards, wherein the laminin reference standards may be laminin isoforms including laminin 111 (α1β1γ1), laminin 211 (α2β1γ1), laminin 311 (α3β1γ1), laminin 411 (α4β1γ1), and laminin 511 (α5β1γ1). Detecting the complex on the substrate may further comprise adding one or more detection agents to the substrate. The one or more detection agents may be horseradish peroxidase enzyme and 3,3′,5,5′-Tetramethylbenzidine. Preparing the micronized mixture may include homogenizing the tissue sample with a homogenization buffer. The homogenization buffer may comprise a mixture of bovine serum albumin and polysorbate 20.

The present disclosure also provides an enzyme-linked immunosorbent assay (ELISA) system for detecting an active laminin protein, the system comprising a capture antibody configured to bind to a beta-1 laminin subunit of the laminin protein; and a detection antibody for binding to a gamma-1 laminin subunit of the laminin protein.

The ELISA system may include one or more of the following aspects. The first antibody may be a laminin beta-1 monoclonal antibody and the second antibody may be a laminin gamma-1 monoclonal antibody. At least one detection agent chosen from horseradish peroxidase enzyme and 3,3′,5,5′-Tetramethylbenzidine. A homogenization buffer comprising a mixture of bovine serum albumin and polysorbate 20. A microplate reader.

The present disclosure also provides a kit for carrying out any of the methods herein.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula does not mean that it cannot also belong to another generic formula.

### BRIEF DESCRIPTION OF THE DRAWINGS

The following drawings form part of the present application and are included to further demonstrate certain aspects of the present disclosure. The disclosure may be better understood by reference to one or more of these drawings in combination with the detailed description of exemplary embodiments presented herein.

FIG. 1 is a schematic view of a laminin protein, as discussed in the present disclosure.

FIG. 2 depicts an exemplary sandwich ELISA assay, according to embodiments of the present disclosure.

FIGS. 3A and 3B present exemplary methods, according to embodiments of the present disclosure.

### DETAILED DESCRIPTION

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, like “about,” “approximately,” or “substantially” are used to indicate a possible variation of ±20% of a stated or understood value, unless other ranges of possible variations are indicated herein. In addition, the term “between” used in describing ranges of values is intended to include the minimum and maximum values described herein.

As used herein, “antigen” and “antibody” are to be taken in their broadest context. An “antigen” can be, for example, any molecule, cell, virus, or particle that may provoke an immune response. For example, an antigen includes, but is not limited to, a chemical molecule, a peptide molecule, a protein molecule, an RNA molecule, a DNA molecule, a traditional antibody, e.g., two heavy chains and two light chains, a recombinant antibody or fragment, a bacterial cell, a virus particle, a cell, a particle, and a product comprising crosslinking any two or more of the above. An antigen can be in a pure form, or it can exist in a mixture. An antigen can be in a modified form (e.g., modified by chemicals) or can be in an unmodified form.

Reference herein to an “antibody” is to be taken in its broadest context. “An antibody” is a polypeptide, such as a protein, that binds to “an antigen.” An antibody includes, but is not limited to, a traditional antibody, a fragment of a traditional antibody containing an antigen binding site, a recombinant antibody containing an antigen binding site, a protein which binds to an antigen, and a product comprising crosslinking any two or more of the above. An antibody can be in a pure form, or it can exist in a mixture. An antibody can be in a modified form (e.g., modified by a chemical) or can be in an unmodified form.

As used herein, the term “homogenous” or “homogenized” may be used more broadly to refer to a mixed sample that does not require every portion of a homogenized sample to be identical throughout.

Tissue grafts and other tissue-based products, for example, nerve tissue grafts, require biological activity in order to function appropriately when implanted into a recipient. Yet previously, the specific components that caused a tissue graft, such a nerve graft, to be biologically active were not fully understood. Once a tissue graft had been prepared, there may have been no robust way to determine the potency of the graft prior to implantation. Active components in a tissue graft that would result in or promote regeneration were not fully understood, as in the case of regeneration of nerve grafts. This disclosure represents an advancement in the art by identifying the active components in tissue grafts, e.g., nerve grafts, that promote regeneration and providing an analytical method for evaluating the presence of and biological activity of the active components. Embodiments of the disclosure present a new method of detecting and quantifying the active components in tissue grafts.

In general, embodiments consistent with the present disclosure may relate to systems and methods for analyzing tissue to determine the presence of and quantity of active laminin components. A variety of tissue types may be analyzed. For example, according to an illustrative example embodiment, the disclosed systems and methods may be utilized for analyzing nerve tissue (peripheral nerve tissue or central nervous system tissue), e.g., which may be subsequently used for in vivo implantation (and/or any other suitable use). In various additional and/or alternative embodiments, the disclosed systems and methods may be utilized for analyzing active laminin components in other tissues, including, but not limited to, epithelial tissue, connective tissue, muscular tissue (e.g., striated and/or smooth muscle tissue), vascular tissue (e.g., capillary tissue), dermal tissue, skeletal tissue (e.g., bone, cartilage, ligaments, fascia, tendons), cardiac tissue, urological tissue (e.g., bladder wall, ureter tissue, urethra tissue), skin tissue, dura tissue, intestinal tissue, gingiva tissue, or adipose tissue, for example. As mentioned above, the soft biological tissue may be mammalian tissue, including, but not limited to, human tissue and tissue of other primates, rodent tissue, equine tissue, canine tissue, rabbit tissue, porcine tissue, or ovine tissue. In addition, the tissue may be non-mammalian tissue, selected from piscine, amphibian, or insect tissue. The tissue may be a synthetic tissue, such as, but not limited to, laboratory-grown tissue or 3D-printed tissue. According to some examples, the tissue is nerve tissue obtained from an animal, such as a human or a non-human mammal. A suitable tissue graft may be a nerve graft, for example, a peripheral nerve graft. The tissue graft, e.g., nerve graft, may have been harvested from a donor (e.g., from a human or animal cadaver), and may have previously been decellurized, cleaned, sterilized, or otherwise preserved. For example, a suitable tissue sample to be tested may be Axogen’s Avance® Nerve Graft, or a similar nerve graft. In some example embodiments, the tissue may include harvested human nerve tissue, e.g., which may be intended for use in nerve repair and/or restoration procedures. It will be appreciated that, in addition and/or as an alternative to human nerve allograft tissue, the systems and methods herein may also be utilized in connection with animal nerve tissue, as well as various other synthetic tissues. Although the disclosure and examples pertain to the analysis of nerve tissue, in particular, any suitable tissue may be analyzed, including those listed above.

Tissue includes various active components, including, e.g., laminin. Laminins, a family of extracellular matrix glycoproteins, are the major active components of the basal lamina (one of the layers of the basement membrane) that represents the adhesive stimulus for successful axonal regeneration. Wang, G Y et al. [1992] Brain Res 570:116-125. Laminin is essential for basement membrane assembly, function, and cell adhesion, and is related to in vitro neurite extension, wherein laminin is the most potent promoter of neurite growth, as compared to other basement membrane proteins. Pulido, 2017; Belkin, 2000; Plantman et al., 2008. Laminins are implicated in a wide variety of biological processes, including cell adhesion, differentiation, migration, signaling, neurite outgrowth, and metastasis.

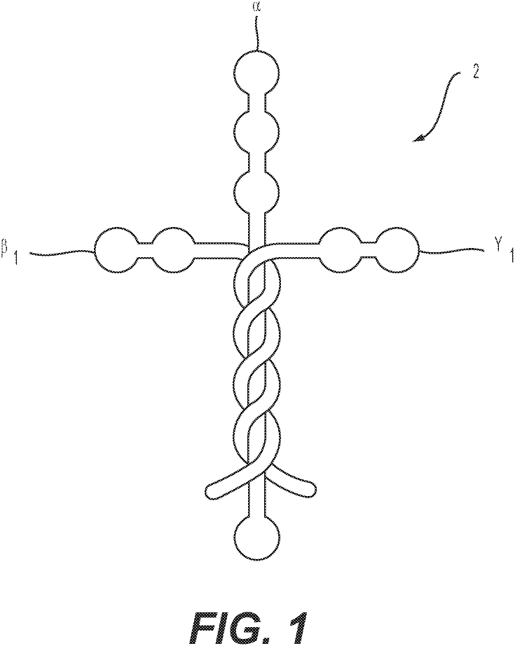

Referring to FIG. 1, a laminin protein 2 is composed of three, non-identical short-arm chains, laminin alpha chain, laminin beta chain, and laminin gamma chain, and a long-arm chain comprised of all three chains. Isoforms of each chain have been discovered, wherein different alpha, beta, and gamma chain isomers combine to produce different laminin isoforms. The laminin isoforms may differ with respect to their tissue distribution. While some isoforms contribute to biological activity, e.g., promote nerve regeneration, other isoforms do not affect biological activity. Accordingly, detection of laminin generally in a tissue may not be sufficient to assess the biological activity of that tissue. Additionally, the presence of laminin isoforms depends on the type of tissue. In other words, different tissues may include different combinations of laminin isoforms.

Embodiments of the disclosure provide an analytical method for evaluating the presence of and detection of the active laminin isoforms, e.g., laminin isoforms containing the beta-1 subunit. Embodiments of the disclosure also enable evaluation of the presence of the active laminin tertiary structure. The methods and systems of the present disclosure depend on the interaction of two of the three laminin chains (the beta and gamma chains), to assess the presence of active laminin in tissue, e.g., nerve tissue. The detection of two of the three laminin chains confirms the presence of the laminin tertiary structure. The beta-1 subunit is found in most tissues that produce basement membranes. The presence of active laminin isoforms containing the beta-1 subunit with intact tertiary structure in tissue, e.g., nerve tissue, signals viability of the tissue, and the systems and methods discussed herein may aid in detecting active components containing the beta-1 subunit with intact tertiary structure in tissue, e.g., human peripheral nerve tissue. Detecting the presence of certain isoforms in tissue may help in the evaluation of regenerative activity of tissue, e.g., neuro-regenerative activity of peripheral nerve tissue. This may allow for quality control and assessment of tissue grafts, for example, nerve grafts. Embodiments of the disclosure may also be used to screen for high-quality donor tissue, for example, human or animal nerve tissue, including, selection of grafts configured to promote an improved outcome for the subject in which the grafts are implanted. Embodiments may also be used to evaluate the presence of active beta-1 subunit in synthetic materials, as well.

Different tissue types may have different active laminin variants. Active laminin variants in different tissues may include combinations of one or more of laminin 111 (α1β1γ1), laminin 211 (α2β1γ1), laminin 311 (α3β1γ1), laminin 411 (α4β1γ1), and laminin 511 (α5β1γ1). Some of these laminin isoforms are present in nerve tissue endoneurial tubes, and more specifically, in fascicles and the basement membrane of the nerve tissue. In the case of nerve tissue, axons only grow within the endoneurial tubes, and thus the presence of the active forms of laminin in these endoneurial tubes may promote, or be responsible for promoting, nerve regeneration. Accordingly, for a nerve graft, such as a peripheral nerve graft, to be potent, the graft may need to include one or more of the 111, 211, 311, 411, and 511 active laminin isoforms. Embodiments of the present disclosure are drawn to an ELISA designed specifically to detect the presence of and/or quantify the amount of active laminin beta-1 subunit in a tissue sample. Accordingly, embodiments of the disclosure may be used to detect the presence of and/or quantify the amount of one or more of active laminin isoforms 111, 211, 311, 411, and 511 in tissue, e.g., nerve tissue, including peripheral nerve tissue grafts.

The purpose of any ELISA is to detect the presence of a target antigen in a sample. In an ELISA, antigens from the sample to be tested are attached to a surface, e.g., a well plate. Then, a matching antibody is applied over the surface so it can bind to the antigen. This matching antibody is linked to an enzyme and then any unbound antibodies are removed, e.g., by washing. A substance containing the enzyme’s substrate is then added to the surface. Any binding between the antigen and enzyme should produce a detectable signal, e.g., a color change in the liquid samples.

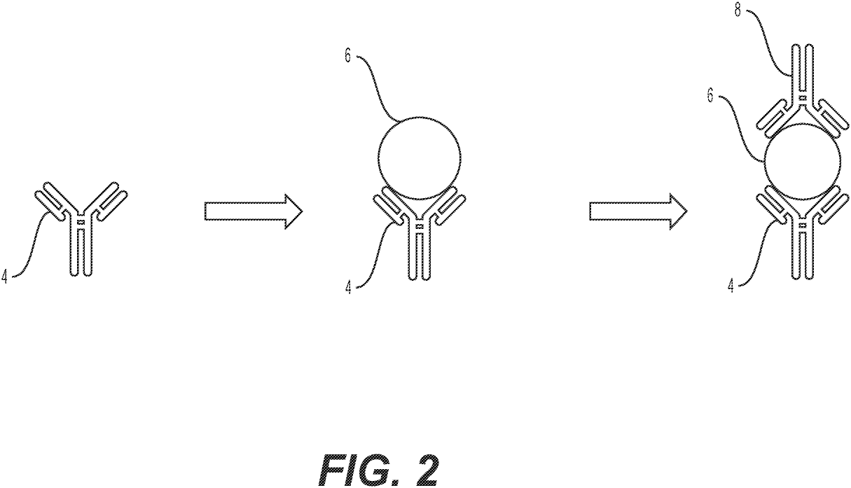

A sandwich ELISA is a type of assay that uses two antibodies: a capture antibody and a detection antibody. In a sandwich ELISA, as shown in FIG. 2, a capture antibody 4 is immobilized to a surface, e.g., a well plate. The surface is then coated with a sample containing a target antigen 6, which binds to the capture antibody 4. Lastly, a detection antibody 8 is added to the surface, which binds to the target antigen 6, allowing for detection of the target antigen 6. The target antigen 6 is thus bound between the capture antibody 4 and the detection antibody 8, sandwiching the target antigen 6 between the two antibodies. Capture antibody 4 and the detection antibody 8 may be selected to detect different epitopes on the target site so as to not interfere with each antibody’s binding capability. Accordingly, the target antigen may contain at least two antigenic sites capable of binding to one of the two antibodies.

Embodiments of the present disclosure are configured to detect the presence and quantity of active forms of laminin in various tissue types. Embodiments of the disclosure may include a capture antibody 4 and a detection antibody 8, where each antibody is configured to bind to a different laminin chain, and the bound capture antibody 4 and detection antibody 8 do not inhibit each other when bound to the laminin chains. Embodiments of the disclosure, e.g., the capture antibody and detection antibody, may depend on the simultaneous interaction with the laminin chains of interest and their ability to not denature or alter the structure of the laminin chains when bound. Without the proper laminin tertiary structure, the ELISA may not be able to detect the active laminin isoforms.

Depending on the tissue type, various aspects and components of the embodiments as discussed herein may be altered or modified. For example, embodiments of the present disclosure are configured to detect the presence and/or quantity of active forms of laminin containing the beta-1 subunit with intact tertiary structure, including one or more of active laminin isoforms 111, 211, 311, 411, and 511. Embodiments of disclosure may include a capture antibody 4 and a detection antibody 8, each configured to bind to one of the beta-1 chain and the gamma-1 chain, wherein the bound capture antibody 4 and detection antibody 8 do not inhibit each other when bound to the beta-1 chain and the gamma-1 chain. Embodiments of the disclosure may depend on the simultaneous interaction with two of the three laminin chains (alpha, beta, and gamma, in this case, beta and gamma) to ensure the presence of the laminin tertiary structure. Additionally, any antibodies used in the ELISA may be selected so as to not denature, or otherwise alter the structure of, the laminin chains. Without the proper laminin tertiary structure, the ELISA may not be able to detect the active laminin isoforms.

In some embodiments, the capture antibody 4 may be configured to bind to the beta-Y subunit, and the detection antibody 8 may be configured to bind to the gamma-1 subunit. In such embodiments, the antibody configured to bind to the beta-1 subunit may be immobilized on the well plate. Next, the substrate with the target antigen, e.g., a micronized tissue sample, such as a nerve tissue sample, may be added to the well plate, and the beta-1 subunit of the laminin target antigen may bind to the capture antibodies. As used herein, the terms “micronized tissue sample” and “homogenized tissue sample” may be used interchangeably herein to refer to a mixture of micronized pieces of tissue mixed with a liquid solution, and the terms do not require that the tissue sample be identical throughout. Then, the detection antibody 8 configured to bind to the gamma-1 subunit may be added onto the antigen, and detection antibody 8 may bind to the gamma-1 subunit of the bound laminin target antigen to allow for detection of the target antigen.

Various amounts of the antibodies, capture antibody 4 and detection antibody 8, may be utilized. For example, the antibody configured to bind to the beta-1 subunit, which may be capture antibody 4, may be applied to the well plate in an amount ranging from about 0.1 µg/mL (antibody to tissue sample) to about 2.0 mg/mL, e.g., about 0.2 µg/mL to about 1.5 mg/mL, or about 0. 5 µg/mL to about 1.0 mg/mL. Detection antibody 8, which may be configured to bind to the gamma-1 subunit, may be applied to the well plate in an amount ranging from about 0.01 µg/mL (antibody to tissue sample) to about 1.0 mg/mL, e.g., about 0.01 µg/Ml to about 0.5 mg/mL. The concentration of antibodies may vary depending, e.g., on the incubation times utilized and the type of tissue analyzed.

In embodiments in which the capture antibody is configured to bind to the gamma-1 subunit and the detection antibody is configured to bind to the beta-1 subunit, the antibody configured to bind to the gamma-1 subunit may be immobilized on the well plate. Next, the substrate with the target antigen, e.g., a homogenized tissue sample, such as a nerve tissue sample, may be added to the well plate, and the gamma-1 subunit of the laminin target antigen may bind to the capture antibodies. Then, the detection antibody 8 configured to bind to the beta-1 subunit may be added onto the antigen, and detection antibody 8 may bind to the beta-1 subunit of the laminin target antigen to allow for detection of the target antigen.

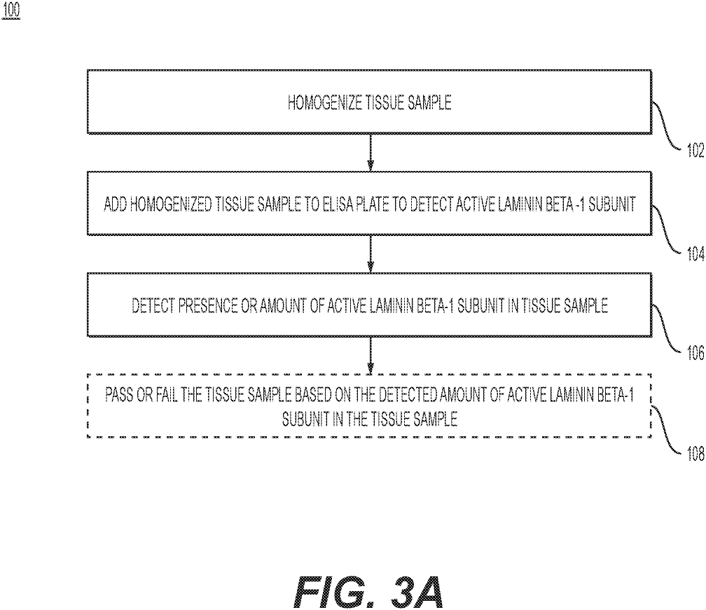

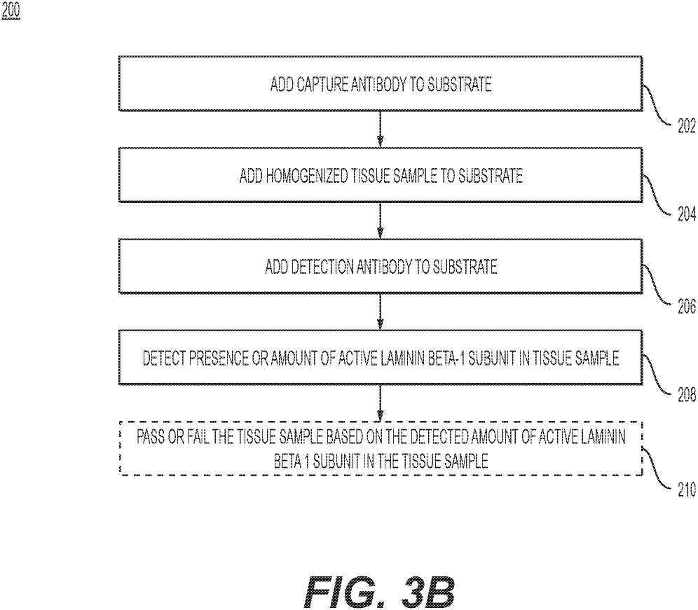

FIGS. 3A and 3B are flow charts showing methods 100, 200 of conducting an ELISA for detection of laminin isoforms having an active beta-1 subunit with intact tertiary structure, e.g., active laminin isoforms 111, 211, 311, 411, and 511. At step 102 of method 100, a tissue sample may be prepared for use. A tissue sample suitable for embodiments of the present disclosure may be natural or synthetic. For example, the tissue may be soft biological tissue obtained from an animal, such as a mammal, including a human or a non-human mammal, or a non-mammal, including a fish, amphibian, or insect. The tissue may be nerve tissue, including, for example, peripheral nerve tissue or central nervous system tissue. Other types of tissue suitable for the present disclosure include, but are not limited to, epithelial tissue, connective tissue, muscular tissue (e.g., striated and/or smooth muscle tissue), vascular tissue (e.g., capillary tissue), dermal tissue, skeletal tissue (e.g., bone, cartilage, ligaments, fascia, tendons), cardiac tissue, urological tissue (e.g., bladder wall, ureter tissue, urethra tissue), skin tissue, dura tissue, intestinal tissue, gingiva tissue, or adipose tissue, for example. As mentioned above, the soft biological tissue may be mammalian tissue, including, but not limited to, human tissue and tissue of other primates, rodent tissue, equine tissue, canine tissue, rabbit tissue, porcine tissue, or ovine tissue. In addition, the tissue may be non-mammalian tissue, selected from piscine, amphibian, or insect tissue. The tissue may be a synthetic tissue, such as, but not limited to, laboratory-grown tissue or 3D-printed tissue. According to some examples, the tissue is nerve tissue obtained from an animal, such as a human or a non-human mammal. A suitable tissue graft may be a nerve graft, for example, a peripheral nerve graft. The tissue graft, e.g., nerve graft, may have been harvested from a donor (e.g., from a human or animal cadaver), and may have previously been decellularized, cleaned, sterilized, or otherwise preserved. For example, a suitable tissue sample to be tested using an ELISA according to the current disclosure may be Axogen’s Avance® Nerve Graft, or a similar nerve graft. As mentioned above, different steps of the methods and/or components utilized in the methods discussed herein may be altered depending on the tissue type.

Preparation of the tissue sample may include liquefying and micronizing a sample tissue graft. The tissue samples may be prepared by any conventional means, e.g., micronization using a MP Biomedicals tissue and cell homogenizer. Prior to use, the cell homogenize may be cooled to a desired temperature, for example, by loading the homogenizer with dry ice. Once the tissue is loaded onto the homogenizer, the homogenizer may be initiated at a speed of about 6 m/s for about 60 seconds. This step may be repeated multiple times. A homogenization buffer may also be added to the micronized tissue sample. An example homogenization buffer is described further below. The micronized tissue may be used in an ELISA shortly after preparation, or may be stored for future testing. If stored, the micronized tissue sample may be stored at approximately room temperature or refrigerated, for example, from about 3° C. to about 25° C., e.g., about 22° C. In some aspects, the micronized tissue sample may be stored at about room temperature to about 0° C. In some aspects, the tissue sample may be stored, for example, at approximately 0° C. to approximately -80° C., at approximately 0° C. to approximately -40° C., at approximately 0° C. to approximately -20° C., or at lower than approximately -80° C., until the tissue samples are utilized in an ELISA. For example, a micronized tissue sample may be stored at approximately room temperature, at approximately 0° C., at approximately -20° C., at approximately -40° C., at approximately -80° C., or lower. The tissue sample may or may not be frozen during storage. If frozen, the micronized tissue sample may be subjected to one or more cycles of freezing and thawing, e.g., 1, 2, 3, 4, 5, 6, or more cycles of freezing and thawing.

Further, if stored, the micronized tissue sample may be stored for a few minutes, a few hours, or a few days. For example, the micronized tissue sample may be stored for about 30 minutes to about 48 hours, from about 30 minutes to about 2 hours, from about 2 hours to about 24 hours, from about 4 hours to about 12 hours, from about 6 hours to about 24 hours, from about 12 hours to about 24 hours, or from about 24 hours to about 48 hours, or more. In some aspects, the tissue sample may be stored for about 2 hours, about 4 hours, about 6 hours, about 12 hours, about 24 hours, about 36 hours, or about 48 hours, or longer. The duration of storage may depend, at least in part, on the temperature of the storage unit and how such temperature may affect the stability of the samples. For example, the micronized tissue samples may be stored at a lower temperature for a longer duration of time, and alternatively, at a higher temperature for a shorter duration of time. In some aspects at temperatures, e.g., of -20° C. or lower, the micronized tissue sample may be stable for up to 6 months or longer, whereas at room temperature, micronized samples may be stable on the order of days.

At step 104, 204, the micronized tissue sample may be added to a substrate, e.g., an ELISA well plate, in order to detect active laminin beta-1 subunit with intact tertiary structure, for example, active laminin isoforms 111, 211, 311, 411, and 511. The micronized tissue may be nerve tissue, for example, derived from a nerve graft. The ELISA plate may be coated with the capture antibody. In method 100, the micronized tissue sample may be added to an ELISA plate that has previously been coated with the capture antibody. In method 200, step 202 of adding a capture antibody to the substrate, e.g., an ELISA well plate, may be performed before step 204. The capture antibody may be configured to bind to one of the beta-1 laminin subunit or the gamma-1 laminin subunit (if present) in the micronized tissue sample added to the well plate. In some aspects of methods 100, 200, a blocking buffer, which may be configured to block the remaining protein-binding sites, may also be added to the substrate, either before or after the addition of the micronized tissue sample.

After the micronized tissue sample is added to the ELISA plate, in method 100, step 106 may be performed to detect a presence or amount of active laminin beta-1 subunit in the micronized tissue sample. This may be done, for example, as specified in method 200, by adding a detection antibody to the ELISA plate at step 206. The detection antibody may be configured to bind to one of the beta-1 laminin subunit or the gamma-1 laminin subunit, whichever subunit the capture antibody is not configured to bind to. For example, if the capture antibody is configured to bind to the beta-1 subunit, then the detection antibody may be configured to bind to the gamma-1 subunit. If the capture antibody is configured to bind to the gamma-1 subunit, then the detection antibody may be configured to bind to the beta-1 subunit.

In either method 100 or method 200, the ELISA plate may be incubated after one or more of addition of the capture antibody to the well plate, addition of the micronized tissue sample to the well plate, or addition of the detection antibody to the well plate, or a combination thereof. For example, after the capture antibody is added to the substrate, the substrate may be incubated for a duration of time ranging from about 5 minutes to about 72 hours, e.g., from about 5 minutes to about 0.5 hours, from about 0.5 hour to about 1 hour, 0.5 hour to about 1.5 hours, about 0.5 to about 2 hours, or about 2 hours to about 6 hours. After the capture antibody is added to the substrate, the substrate may be incubated for a duration of time ranging from about 6 hours to about 15 hours, about 16 hours to about 24 hours, about 15 hours to about 72 hours, about 24 hours to about 72 hours, about 24 hours to about 36 hours, about 36 to about 72 hours, or longer than 72 hours. The substrate may be incubated at a temperature ranging from about 1° C. to about 37° C. In some aspects, once the micronized tissue sample has been added to the ELISA plate, e.g., before the addition of the detection antibody or after the addition of the detection antibody, the substrate may be incubated. For example, the micronized tissue sample may be incubated for about 15 minutes to about 2 hours, from about 30 minutes to about 1.5 hours, from about 30 minutes to about 1 hour, from about 40 minutes to about 1 hour, or about 45 minutes to about 1 hour after the addition of the tissue sample to the capture antibody and/or the addition of the detection antibody to the ELISA.

In either method 100 or method 200, one or more washing steps may be performed to remove unbound components, e.g., after each incubation step. For example, one or more washing steps may be performed after binding a capture antibody to the substrate, after the addition of the micronized tissue sample to the substrate, or after addition of a detection antibody to the substrate. In some embodiments, the solid substrate may be washed prior to detection of the detection antibody. Washing the solid substrate prior to detection of the detection antibody may promote removal of unbound detection antibody, which may decrease the level of background signal and hence improve the sensitivity of the ELISA. Methods for washing steps are known in the art and generally involve one or more additions and removal of a buffer solution. Accordingly, the substrate may be washed with a buffer solution. For example, the substrate may be washed with tris-buffered saline (TBS), tris-buffered saline and polysorbate 20 (TBST), phosphate-buffered saline (PBS), or any appropriate buffer solution. After the washing is performed, the substrate may be dried, for example, by blotting with an absorbent medium (e.g., a paper towel), or any other suitable drying method. In some aspects, other types of polysorbate, e.g., polysorbate 40, polysorbate 60, or polysorbate 80, may be used instead of, or in addition to, polysorbate 20.

The antibodies utilized in the ELISA, e.g., the capture antibody and detection antibody, may be present in various reagents. The reagents used in the systems and methods of the present disclosure may depend on the laminin chains of interest. Embodiments of the present disclosure may utilize reagents chosen from anti-laminin beta antibodies and anti-laminin gamma antibodies. For example, the capture antibody may be an anti-laminin beta antibody, and the detection antibody may be an anti-laminin gamma antibody, or vice versa. The anti-laminin beta antibody may be a laminin beta-1 antibody, e.g., mouse anti-human laminin beta-1 monoclonal antibody (available from Millipore Sigma), or rat 4E10 anti-laminin beta-1 antibody (available from Millipore Sigma). The anti-laminin gamma antibody may be a laminin gamma-1 antibody, e.g., biotinylated anti-h/r laminin gamma-1 Purified Mouse Monoclonal IgG, or a conjugated human/rat laminin gamma-1 antibody (available from R&D Systems). The concentration of and/or type of antibodies and/or reagents utilized with the antibodies may be determined depending on the tissue type to be analyzed and laminin isoforms to be identified.

Controls may also be run with an ELISA. Running the appropriate controls may help to accurately distinguish between true positive results and false results. Controls may be positive controls or negative controls. Positive controls may use an endogenous soluble sample known to contain the target analyte, or a purified protein or peptide known to contain an immunogenic sequence for the antibody utilized in the ELISA. A positive result from the positive control may indicate the procedure is optimized and working, and will verify that any negative results are valid. A negative control is a sample that is known to not express the target analyte. Using a negative control may check for non-specific binding and false positive results. In some examples, a standard may also be utilized in the ELISA. The standard is a sample that contains a known concentration of the target analyte from which the standard curve may be obtained. A poor standard curve may mean the antibody did not bind properly or does not capture the protein standard. Controls and samples may be tailored to the specific type of tissue to be tested based, e.g., on the types of active laminin that may be present in that tissue and thus the types of active laminin to be detected in the sample tissue. For example, an assay may use a standard curve that is made of the laminin isoforms that are expected to be present in that type of tissue.

Referring back to FIGS. 3A and 3B, step 106 may further include using a qualitative or quantitative inspection to detect the presence or amount of active laminin beta-1 subunit in the micronized tissue sample. For example, in method 200, after the capture antibody, micronized tissue sample, and detection antibody have been added to the substrate, the detection step 208 may be performed. For example, during steps 106, 208, detection enzymes, or other suitable detection agents, may be applied to the substrate. Enzymes used for detection in ELISA include, for example, horseradish peroxidase enzyme (HRP, e.g., Poly-HRP Streptavidin, available from Thermo Fisher Scientific). In order to produce a color change, a substrate for the HRP may be added. For example, 3,3′,5,5′-Tetramethylbenzidine (TMB) is a substrate for HRP and is a label conjugated to antibodies for applications such as immunohistochemistry, western blotting, and ELISA. The combination of HRP and hydrogen peroxide may cause the oxidation of TMB, resulting in a color change, e.g., in the formation of a yellow or blue color. One or more additional solutions, e.g., a stopping solution, may be added to the samples in order to stop the reaction of the HRP and TMB. In an embodiment of the present disclosure, the stopping solution may be sulfuric acid, although any suitable stopping solution may be used.

At steps 106, 208, after the substrate has been prepared, the colorimetric signals, i.e., color change, of the samples, may be read in an appropriate qualitative or quantitative manner, e.g., using visual inspection, a spectrophotometer, microplate reader, or other suitable ELISA plate reader. The results may then be analyzed to determine the presence or concentration of laminin within the micronized tissue of interest. Qualitative reads (e.g., to determine a presence of the active laminin beta-1 subunit), quantitative reads (e.g., to determine an amount of the active laminin beta-1 subunit), or a combination of both may be performed. In some embodiments, methods 100, 200 may be used to determine whether or not the active laminin beta-1 subunit has been detected in the sample. In this instance, a binary outcome (present or not present) may be sufficient. In other embodiments, method 100, 200 may be performed to determine the quantity, e.g., concentration, of active laminin beta-1 subunit in the sample. In some aspects, once the presence or quantity of active laminin isoforms is determined, methods 100, 200 may end. For example, if method 100, 200 is to be used to evaluate the presence of active beta-1 chain in a tissue sample, or the concentration of active beta-1 chain, the methods may end after steps 106, 208.

In some embodiments of the disclosure, however, methods 100, 200 may proceed to optional steps 108, 210. At steps 108, 210, the concentration of active beta-1 chain detected may be used to pass or fail the tissue sample based on the determined amount of active laminin beta-1 subunits detected in the tissue sample. For example, the detected concentration may be compared to a pre-determined threshold concentration level. If the detected concentration of active laminin beta-1 subunits in the tissue sample meets or exceeds this threshold, then the sample may be indicated as having passed. If the detected concentration of active laminin beta-1 subunits in the tissue sample falls below the pre-determined threshold, then the sample may be indicated as having failed. In some aspects, detecting the presence of any active laminin beta-1 subunits may be sufficient for the tissue sample to be indicated as having passed, whereas failing to detect any active laminin beta-1 subunits may result in the tissue sample being indicated as having failed.

Inclusion of steps 108, 210 in methods 100, 200 may allow for quality control and assessment of sample tissue, such as tissue grafts, for example, nerve grafts. Embodiments of the disclosure may also be used to screen for high-quality donor tissue, for example, human or animal nerve tissue. Embodiments may also be used to screen for inclusion of active laminin beta-1 subunits in synthetic materials, as well. This method may be performed at any point during the tissue acquisition, tissue preparation, tissue storage, tissue shipping, quality control, or end-use process, for example, during or following the harvesting of tissue, during of following creation of a synthetic tissue, during or following the decellularization of, recellularization of, or other processing of a tissue, during or following packaging of a tissue for storage or shipment, e.g., after packaging of a tissue and before shipment, during or after storage of a tissue, prior to implantation of a tissue, after receipt of a tissue from a manufacturer, or at any other suitable time point.

Additional components may be utilized in the systems and methods of the present disclosure. Such components may include reagents, solvents, buffers, supplements, chelating agents, and enzymes. For example, the additional components may be chosen from water, protease inhibitor, protein concentration standards, e.g., serum albumin proteins (Bovine Serum Albumin (BSA) available from Millipore Sigma), digestive enzymes (alpha-Chymotrypsin), fetal bovine serum, protease inhibitors, trypsin (0.25% Trypsin-EDTA available from ThermoFisher Scientific), or combinations thereof.

The following examples are included to demonstrate exemplary embodiments of the disclosure without, however, being limiting in nature. It is understood that the present disclosure encompasses additional embodiments consistent with the foregoing description and following examples. It should be appreciated by those of skill in the art that the techniques disclosed in the examples that follow represent techniques discovered by the inventors to function well in the practice of the disclosure, and thus can be considered to constitute exemplary modes for its practice. However, those of skill in the art should, in light of the present disclosure, appreciate that many changes can be made in the specific embodiments which are disclosed and still obtain a like or similar result without departing from the spirit and scope of the disclosure.

In the following examples, the presence of active laminin beta-1 subunit in a nerve tissue sample, specifically, a subset of the laminin 111, laminin 211, laminin 311, laminin 411, and laminin 511 isoforms, was determined using a sandwich immunoassay. A capture antibody was immobilized in a 96-well plate, laminin isoforms having an active beta-1 subunit were bound to the capture antibody, and a horseradish peroxidase conjugated secondary detection antibody was used to generate an absorbance response that was directly proportional to the amount of laminin having an active beta-1 subunit detected in the well. Although specific parameters, e.g., incubation times, temperature ranges, etc., are described in the examples below for proof of concept, it is contemplated that different parameters may be used to perform this ELISA. For example, the capture antibody may be coated onto the plate and allowed to sit for several minutes, several hours, several days, or longer prior to addition of the micronized tissue sample. Antigen, e.g., a component of the micronized tissue sample, may be allowed to sit for several minutes, several hours, or several days, after the tissue sample has been added to the plate containing the immobilized capture antibody. The detection antibody may be allowed to sit for several minutes, several hours, or several days, as described above, after addition to the micronized tissue sample.

### Example 1

### Preparation of Homogenization Buffer

Homogenization buffers were prepared as follows. 2 g ± 0.02 g of bovine serum albumin (BSA) was loaded into a sterile storage bottle, and 180 mL of cell culture water and 20 mL of polysorbate 20 (10X Tris Buffered Saline - Tween (TBST)) was added. 2 mL of a protease inhibitor ( P2714-1BTL-PW from Millipore Sigma) was added to the solution. For a minimum of 30 minutes, the sterile storage bottle was gently swirled and occasionally inverted to fully dissolve the BSA.

### Example 2

### Preparation of Blocking Buffer

Blocking buffers, also referred to as a tris-buffered saline bovine serum albumin (TBS-BSA) block, were prepared as follows. 2 g ± 0.02 g of BSA was loaded into a sterile storage bottle, and 180 mL of cell culture water and 20 mL of 10X TBS was added. For a minimum of 30 minutes, the sterile storage bottle was gently swirled and occasionally inverted to fully dissolve the BSA.

### Example 3

### Preparation of Assay Diluent

Assay diluents were prepared as follows. 2 g ± 0.02 g of BSA was loaded into a sterile storage bottle, and 180 mL of cell culture water and 20 mL of 10X TBST was added. For a minimum of 30 minutes, the sterile storage bottle was gently swirled and occasionally inverted to fully dissolve the BSA.

### Example 4

### Preparation of 1X Tris-Buffered Saline (TBS)

1X TBS was prepared as follows. 180 mL of cell culture water and 20 mL of 10X TBS was added to a sterile storage bottle. The sterile storage bottle was gently swirled to mix the components.

### Example 5

### Preparation of 1X TBST Wash Buffer

1X TBST wash buffers were prepared as follows. 1800 mL of cell culture water and 200 mL of 10X TBST was added to an appropriately sized container.

### Example 6

### Preparation of Protease Inhibitor

Protease inhibitors were prepared as follows. 10 mL of cell culture water was pipetted into the vial of a lyophilized protease inhibitor. The vial was inverted a few times to fully dissolve the protease inhibitor. 1 mL aliquots of protease inhibitor solution were added into 1.5 mL tubes.

### Example 7

### Preparation of Laminin Reference Standards

Laminin reference standards were prepared as follows. Prior to use, the laminin reference materials were thawed, aliquoted, and frozen at -80° C. for a minimum of 12 hours. The laminin reference materials were chosen from three laminin isoforms, referred to herein as laminin A, laminin B, and laminin C. Each of the three standards underwent at least 1 freeze-thaw cycle, although additional freeze-thaw cycles may be undergone, e.g., 2, 3, 4, or more. For each assay, a standard pre-dilution was prepared by mixing laminin A, laminin B, and laminin C, at a ratio of 1:1:2, respectively, into a homogenization buffer (as prepared in Example 1 above) containing 20% negative control (NC) for a total laminin concentration of 10 µg/mL (10,000 ng/mL). NC is nerve tissue material without any active laminin isoforms. The pre-dilution reference standard preparations are presented in Table 1 below. The NC was negative control nerve tissue that did not contain any of the active components, i.e., did not contain laminin A, laminin B, or laminin C, in the tissue of interest including the laminin protein 2.

TABLE 1

Reference Standard Pre-Dilution Preparation

| Label | Stock Concentration (ng/mL) | Volume of laminin stock (µL) | Volume of 1:5 NC (µL) | Final Concentration (ng/mL) | Total Vol. (µL) |
|---|---|---|---|---|---|
| Laminin A | 100,000 | 5 | 180 | 10,000 | 200 |
| Laminin B | 100,000 | 5 | | | |
| Laminin C | 100,000 | 10 | | | |

Eight of the wells of the plate were filled with assay reference standards at approximately 330 ng/mL, 220 ng/mL, 147 ng/mL, 97.8 ng/mL, 65.2 ng/mL, 43.5 ng/mL, 29.0 ng/mL, and 19.3 ng/mL, of total laminin protein. The eight assay reference standards are presented in Table 2 below. The standard curve was prepared by first preparing the top standard (330 ng/mL) by spiking the appropriate volume of the standard pre-dilution sample into a homogenization buffer (e.g., prepared according to Example 1) containing 20% (v/v) NC (1:5 Negative Control). Each subsequent working reference standard was prepared in a similar homogenization protocol, for a total of eight non-zero, non-anchor points.

TABLE 2

Reference Standard Curve Preparation

| Label | Stock | Stock Concentratio n (ng/mL) | Volume of Stock (µL) | Volume of 1:5 NC (µL) | Final Concentratio n (ng/mL) | Total Vol. (µL) |
|---|---|---|---|---|---|---|
| STD1 | Standard Pre-Dilution | 10,000 | 33 | 967 | 330 | 1000 |
| STD2 | STD1 | 330 | 480 | 240 | 220 | 720 |
| STD3 | STD2 | 220 | 480 | 240 | 147 | 720 |
| STD4 | STD3 | 147 | 480 | 240 | 97.8 | 720 |
| STD5 | STD4 | 97.8 | 480 | 240 | 65.2 | 720 |
| STD6 | STD5 | 65.2 | 480 | 240 | 43.5 | 720 |
| STD7 | STD6 | 43.5 | 480 | 240 | 29.0 | 720 |
| STD8 | STD7 | 29.0 | 480 | 240 | 19.3 | 720 |

### Example 8

### Preparation of System Suitability Samples

System suitability samples (SS samples) were prepared as follows. SS samples were prepared from tissue graft samples positive for laminin beta-1 subunits. The tissue graft samples were cut and homogenized according to any of the protocols discussed herein. An MP Biomedical tissue homogenizer was used to micronized the nerve tissue. The homogenizer was first be loaded with dry ice to cool down the system. The tissue was then loaded into the homogenizer. The tissue was micronized at the speed of about 6 m/s, for about 60 seconds. This step was repeated twice. The micronized SS samples were stored at -80° C. until used in the assay. Each plate contained two sets of system suitability controls. 50 µL of the SS sample was added to 200 µL of homogenization buffer to dilute the SS samples to the minimum required dilution (MRD).

### Example 9

### Preparation of Stock Quality Control Samples

A minimum of three quality control samples (QC) at in-assay total laminin concentrations of 196 ng/mL (high quality control sample, HQC or CTL1), 81 ng/mL (mid quality control sample, MQC or CTL2), and 31 ng/Ml (low quality control sample, LQC or CTL3) were included in each validation run. Stock QC samples were spiked with a 1:1:2 mixture of laminin proteins (25% laminin A, 25% laminin B, and 50% laminin C) at a concentration 5 times (5x) the in-assay concentrations in the micronized negative control nerve tissue. The QC stock, pre-dilution are detailed below in Table 3.

Each QC sample was diluted to a minimum required dilution (MRD, 1:5) in a homogenization buffer to achieve the desired in-assay concentrations as listed below in Table 4. Stock QC samples were prepared by spiking neat NC with the appropriate volume of pre-dilution sample, which contained a mixture of laminin A, laminin B, and laminin C at a 1:1:2: ratio respectively. The stock QC samples were bulk prepared, aliquoted, and stored at -80° C.

TABLE 3

Preparation of Quality Control Stock Pre-Dilution

| Label | Stock Concentration (ng/mL) | Vol. of BioLaminin stock (µL) | Vol. of 1:5 NC (µL) | Final Concentration (ng/mL) | Total Vol. (µL) |
|---|---|---|---|---|---|
| Laminin A | 100,000 | 15 | 540 | 10,000 | 600 |
| Laminin B | 100,000 | 15 | | | |
| Laminin C | 100,000 | 30 | | | |

TABLE 4

Preparation of Quality Control Stocks

| Label | Stock (10,000 ng/mL) | Concentration (ng/mL) | Volume of Stock (µL) | Volume of NC (µL) | Final Volume (µL) |
|---|---|---|---|---|---|
| HQC - stock | Predilution | 980 | 245 | 2255 | 2500 |
| MQC - stock | Predilution | 405 | 105 | 2488 | 2593 |
| LQC - stock | Predilution | 155 | 40 | 2541 | 2581 |

### Example 10

### Preparation of Working Quality Control Samples

Working quality control samples were prepared as follows. The stock QC samples, e.g., as prepared according to the protocol in Example 9, were diluted to the MRD by adding 50 µL of each quality control sample to 200 µL of homogenization buffer, as detailed in Table 5 below. Each QC was prepared in duplicate sets made independent of one another.

TABLE 5

Quality Control Samples

| Label | Stock Label | Stock Concent ration (ng/mL) | Volume of QC (µL) | Volume of Homogenizat ion Buffer (µL) | Final Concentratio n (ng/mL) | Final Volume (µL) |
|---|---|---|---|---|---|---|
| HQC | HQC - stock | 980 | 50 | 200 | 196 | 250 |
| MQC | MQC - stock | 405 | 50 | 200 | 81 | 250 |
| LQC | LQC - stock | 155 | 50 | 200 | 31 | 250 |

### Example 11

### Preparation of Nerve Graft Samples

Nerve Graft Cutting. Avance Nerve Graft samples were removed from -80° C. storage and transported in an ice bucket containing dry ice to a cleaned biosafety cabinet. Once inside the biosafety cabinet, the outer pouch of the samples was peeled open using aseptic technique. The plastic tray inside the foil pouch was then removed, filled with room temperature 1X PBS, and allowed to thaw for 5-10 minutes. In a small container, e.g., a petri dish, 1X PBS was added to the chamber of the cutting board. Prior to use, the razor blades were also soaked in 1X PBS. Once the graft was properly thawed, using sterile Dumont fine forceps, the graft was handled by the outer most epineurium to avoid crimping or crushing the graft. The graft was then placed at the bottom of the cutting board, making sure to extend the graft slightly beyond the first mark by approximately 1 mm. To avoid drying out the graft handle the grafts were moved gently but quickly. Next, razor blades were placed at each end of the graft, after which, a fresh razor blade was positioned in the first space of the cutting board. The blades were then placed 3 mm apart along the length of the nerve graft for small diameter nerves (less than 3 mm in diameter), or the blades were placed 1 mm apart, with 2 mm spaces between the razor blades surrounding the 1 mm segments, along the length of the nerve graft for large diameter nerves (more than 3 mm in diameter). Blades were also partially inserted in the slice channels of the cutting board.

Using a 50 mL conical tube, even pressure was placed on top of all razor blades in the cutting board and pushed down simultaneously into the cavity until the blades could not be pressed further. Each blade was then moved back and forth a few times to completely sever the tissue into 3 mm segments. The sample collection blades were then removed one by one. Next, each segment was added to a 1.5 mL tube containing 100 µL of homogenization buffer. Including a homogenization buffer along with the segments may prevent the segments from drying out during the weight process prior to homogenization. Each segment was then placed on paper, e.g., paper fiber optic cleaning wipes, to soak up excess fluid. The segment was then weighed, and the mass of each segment was recorded.

Each segment must fall within the target mass range of 5.0 mg to 9.0 mg. If the segment was less than 5.0 mg, an additional segment was added to reach the target range. If the segment was greater than 9.0 mg, the segment was cut smaller, e.g., using a razor blade, to bring the segment within the acceptable range. After recording the mass of each segment, the segment was transferred to a homogenization tube containing 600 µL of homogenization buffer.

Homogenization of Nerve Graft Samples. Nerve graft samples were micronized using a conventional grinder and lysis system, e.g., a FastPrep-24® available from MPBio. Additional samples, e.g., the system suitability samples of Example 8, stock quality control samples of Example 9, and/or working quality control samples of Example 10, were also micronized using a conventional grinder and lysis system.

### Loading and Homogenizing Samples

After completion of the homogenization process using the grinder and lysis system, the homogenates of the samples were transferred to cryovials or microcentrifuge tubes and stored at -80° C. until use. To prepare an SS sample, all vials of SS sample homogenate were be pooled, and then 60 µL aliquots were made in microcentrifuge tubes.

### Example 12

### Assay Procedure

An assay according to the systems and methods discussed herein was performed as follows. A nonsterile 96-well plate was coated with a laminin beta capture antibody (Mouse Anti-Human Laminin beta-1 Monoclonal antibody, available from Millipore), at a concentration of 2 µg/mL. 100 µl of the capture anitbody was dispensed into each well of the plate. The plate was then sealed and placed on a shaker at room temperature for 5-10 minutes, at a setting of 200 rpm to 300 rpm, and then incubated at 5° C. for 18-72 hours. After incubation, the plate was washed on an automated plate washer with 300 µL/well of 1X TBST wash buffer, and dried, e.g., via blotting on a paper towel.

300 µl of TBS-BSA block was then added to each well of the plate. The plate was then incubated for 1.5 ± 0.5 hours in an incubator set at 37° C. on a plate shaker at a setting of 200-300 rpm. The reference standards (Example 7), and SS sample (Example 8) = were prepared according to the Examples and procedures described herein. After the incubation period, the plate was washed with 300 µL/well of 1X TBST wash buffer, and dried, e.g., via blotting on a paper towel.

The test samples, SS samples, reference standards, homogenization buffer (used as a blank), and controls diluted to the MRD were all dispensed into the appropriate wells of the plate, at 100 µl/well, and then incubated for 1.5 ± 0.5 hours in an incubator set at 37° C. on a plate shaker at a setting of 200-300 rpm. After the incubation period, the plate was washed three times with 300 µl/well of 1X TBST wash buffer. Any residual wash buffer was tapped out of the plate. Table 6 below details the plate map designating which component was added to each well.

TABLE 6

| | | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| A | STD 1 | SPL 1 Dil 1 | SPL 5 Dil 1 | SPL 9 Dil 1 | HQC -a | HQC -b | STD 1 | SPL 1 Dil 1 | SPL 5 Dil 1 | SPL 9 Dil 1 | HQC-a | HQC -b |
| B | STD 2 | SPL 1 Dil 2 | SPL 5 Dil 2 | SPL 9 Dil 2 | MQC-a | MQC-b | STD 2 | SPL 1 Dil 2 | SPL 5 Dil 2 | SPL 9 Dil 2 | MQC -a | MQC-b |
| C | STD 3 | SPL 2 Dil 1 | SPL 6 Dil 1 | SPL 10 Dil 1 | LQC -a | LQC-b | STD 3 | SPL 2 Dil 1 | SPL 6 Dil 1 | SPL 10 Dil 1 | LQC -a | LQC -b |
| D | STD 4 | SPL 2 Dil 2 | SPL 6 Dil 2 | SPL 10 Dil 2 | NC | NC | STD 4 | SPL 2 Dil 2 | SPL 6 Dil 2 | SPL 10 Dil 2 | NC | NC |
| E | STD 5 | SPL 3 Dil 1 | SPL 7 Dil 1 | SPL 11 Dil 1 | Blank | Blank | STD 5 | SPL 3 Dil 1 | SPL 7 Dil 1 | SPL 11 Dil 1 | Blank | Blank |
| F | STD 6 | SPL 3 Dil 2 | SPL 7 Dil 2 | SPL 11 Dil 2 | SS | | STD 6 | SPL 3 Dil 2 | SPL 7 Dil 2 | SPL 11 Dil 2 | SS | |
| G | STD 7 | SPL 4 Dil 1 | SPL 8 Dil 1 | SPL 12 Dil 1 | | | STD 7 | SPL 4 Dil 1 | SPL 8 Dil 1 | SPL 12 Dil 1 | | |
| H | STD 8 | SPL 4 Dil 2 | SPL 8 Dil 2 | SPL 12 Dil 2 | | | STD 8 | SPL 4 Dil 2 | SPL 8 Dil 2 | SPL 12 Dil 2 | | |

A laminin gamma detection antibody (Biotinylated anti-h/r laminin gamma-1 Purified Mouse Monoclonal IgG Clone D18 Ab-1) at concentration of 0.5 µg/mL, was then added to the wells, at 100 µl/well. After application of the detection antibody, the plate was incubated for 1.5 ± 0.5 hours in an incubator set at 37° C. on a plate shaker at a setting of 200-300 rpm. After this incubation period, the plate was washed three times with 300 µl/well of 1X TBST wash buffer and dried.

A peroxidase enzyme, e.g., Poly-HRP Streptavidin, was diluted to the appropriate working concentration, and 100 µL of diluted peroxidase enzyme was added to the wells of the plate. This dilution may be re-evaluated and updated, if necessary, upon use of different lots of peroxidase enzyme and laminin gamma detection antibody. The plate was incubated for 1.5 ± 0.5 hours in an incubator set at 37° C. on a plate shaker at a setting of 200-300 rpm. Following this incubation period, the plate was washed three times with 300 µl/well of TBST wash buffer and dried.

100 µl of 1:1 diluted peroxidase substrate and peroxide solution was then added to the wells of the plate. The plate was then incubated at room temperature on a plate shaker at a setting of 200-300 rpm for 30 ± 20 minutes. This incubation period may be re-evaluated and updated, if necessary, upon use of different lots of peroxidase enzyme and laminin gamma detection antibody. Upon completion of this incubation period, 100 µl of 2 M Sulfuric Acid was dispensed to all wells of the plate and produed a yellow cover. The plate was then incubated for a minimum of 1 minute at room temperature. The plate was then covered and read on a multiplate reader to measure absorbance at 450 nm. The plate was read within 20 minutes of acidification. The concentration of laminin present in the test samples was calculated from weighted 4-parameter nonlinear logistic (4PL) curve fits using the software Gen5 v3 or later software.

All of the methods disclosed and claimed herein can be made and executed without undue experimentation in light of the present disclosure and the knowledge of one of ordinary skill in this art. While the compositions and methods of this disclosure have been described in terms of exemplary embodiments, it will be apparent to those of skill in the art that variations may be applied to the methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method of performing an enzyme-linked immunosorbent assay (ELISA), the method comprising:
immobilizing a capture antibody to a substrate, wherein the capture antibody is configured to bind to a laminin beta-1 chain;
adding a micronized tissue sample to the substrate containing the capture antibody;
adding a detection antibody to the substrate containing the capture antibody and the micronized tissue sample, wherein the detection antibody is configured to bind to a laminin gamma-1 chain;
detecting whether a complex of the capture antibody, a target antigen in the micronized tissue sample, and the detection antibody has been formed, wherein presence of the complex indicates presence of the target antigen with intact tertiary structure in the micronized tissue sample, and absence of the complex indicates absence of the target antigen with intact tertiary structure in the micronized tissue sample.

2. The method of claim 1, wherein the target antigen includes one or more of laminin 111 (α1β1γ1), laminin 211 (α2β1γ1), laminin 311 (α3β1γ1), laminin 411 (α4β1γ1), and laminin 511 (α5β1γ1).

3. The method of claim 1, wherein the detecting comprises determining a concentration of the target antigen.

4. The method of claim 3, wherein the method further comprises passing or failing the micronized tissue sample based at least in part on the determined concentration of the target antigen.

5. The method of claim 1, wherein the detecting comprises determining a presence of the target antigen.

6. The method of claim 5, wherein the method further comprises passing or failing the micronized tissue sample based at least in part on the determined presence of the target antigen.

7. The method of claim 1, further comprising washing the substrate with a buffer solution chosen from tris-buffered saline, tris-buffered saline and polysorbate 20, phosphate-buffered saline, or mixtures thereof, after one or more of: adding the capture antibody to the substrate, adding the micronized tissue sample to the substrate, or adding the detection antibody to the substrate.

8. The method of claim 1, wherein the detecting is performed using one or more of visual inspection, a spectrophotometer, or a microplate reader.

9. The method of claim 1, further comprising incubating the substrate after one or more of: adding the capture antibody to the substrate, adding the micronized tissue sample to the substrate, or adding the detection antibody to the substrate.

10. The method according to claim 1, wherein the tissue sample is nerve tissue.

11. The method according to claim 1, wherein the tissue sample includes one or more of: human epithelial tissue, connective tissue, muscular tissue, vascular tissue, dermal tissue, skeletal tissue, cardiac tissue, urological tissue, skin tissue, dura tissue, intestinal tissue, gingiva tissue, or adipose tissue.

12. The method according to claim 1, wherein the specimen of tissue includes one or more of: epithelial tissue, connective tissue, muscular tissue, vascular tissue, dermal tissue, skeletal tissue, cardiac tissue, urological tissue, skin tissue, dura tissue, intestinal tissue, gingiva tissue, or adipose tissue.

13. A method of detecting an active laminin protein in a tissue sample, comprising:
preparing a micronized mixture from a tissue sample, wherein the tissue sample contains nerve tissue;
immobilizing a first antibody to the substrate;
adding the micronized mixture to the substrate containing the first antibody, wherein the first antibody is configured to bind to a first antigenic site of the laminin protein if present in the micronized mixture;
adding a second antibody to the substrate containing the first antibody and the micronized mixture, wherein the second antibody is configured to bind to a second antigenic site of the laminin protein if present in the micronized mixture; and
detecting whether a complex of the first antibody, the laminin protein, and the second antibody has been formed, wherein presence of the complex indicates presence of one or more of laminin 111 (α1β1γ1), laminin 211 (α2β1γ1), laminin 311 (α3β1γ1), laminin 411 (α4β1γ1), and laminin 511 (α5β1γ1) isoforms with intact tertiary structure, and absence of the complex indicates absence of one or more of laminin 111 (α1β1γ1), laminin 211 (α2β1γ1), laminin 311 (α3β1γ1), laminin 411 (α4β1γ1), and laminin 511 (α5β1γ1) isoforms with intact tertiary structure.

14. The method of claim 13, wherein the first antigenic site is a beta-1 laminin subunit of the laminin protein.

15. The method of claim 13, wherein the second antigenic site is a gamma-1 laminin subunit of the laminin protein.

16. The method of claim 13, wherein the first antibody is an anti-laminin beta-1 antibody.

17. The method of claim 16, wherein the anti-laminin beta-1 antibody is a mouse anti-human laminin beta-1 monoclonal antibody.

18. The method of claim 13, wherein the second antibody is an anti-laminin gamma-1 antibody.

19. The method of claim 18, wherein the anti-laminin gamma-1 antibody is biotinylated anti-h/r/ laminin gamma-1 Purified Mouse Monoclonal IgG.

20. The method of claim 13, further comprising preparing and analyzing laminin reference standards, wherein the laminin reference standards are laminin isoforms including laminin 111 (α1β1γ1), laminin 211 (α2β1γ1), laminin 311 (α3β1γ1), laminin 411 (α4β1γ1), and laminin 511 (α5β1γ1).

21. The method of claim 13, wherein detecting the complex on the substrate further comprises adding one or more detection agents to the substrate.

22. The method of claim 21, wherein the one or more detection agents are horseradish peroxidase enzyme and 3,3′,5,5′-Tetramethylbenzidine.

23. The method of claim 13, wherein preparing the micronized mixture includes homogenizing the tissue sample with a homogenization buffer.

24. The method of claim 23, wherein the homogenization buffer comprises a mixture of bovine serum albumin and polysorbate 20.

25. An enzyme-linked immunosorbent assay (ELISA) system for detecting an active laminin protein, the system comprising:
a capture antibody configured to bind to a beta-1 laminin subunit of the laminin protein; and
a detection antibody for binding to a gamma-1 laminin subunit of the laminin protein.

26. The system of claim 25, wherein the first antibody is a laminin beta-1 monoclonal antibody, and the second antibody is a laminin gamma-1 monoclonal antibody.

27. The system of claim 25, further comprising at least one detection agent chosen from horseradish peroxidase enzyme and 3,3′,5,5′-Tetramethylbenzidine.

28. The system of claim 25, further comprising a homogenization buffer comprising a mixture of bovine serum albumin and polysorbate 20.

29. The system of claim 25, further comprising a microplate reader.

30. A kit for carrying out the method according to any one of claims 13-24.
